(19) United States
(12) Patent Application Publication
(10) Pub. No.: US 2015/0265449 A1
(43) Pub. Date: Sep. 24, 2015

(54) Ankle airbag device and method for preventing sprain injuries

(71) Applicants: Matteo Togninalli, Verscio (CH); Danilo Togninalli, Verscio (CH)

(72) Inventors: Matteo Togninalli, Verscio (CH); Danilo Togninalli, Verscio (CH)

(21) Appl. No.: 14/661,699

(22) Filed: Mar. 18, 2015

Related U.S. Application Data
(60) Provisional application No. 61/969,280, filed on Mar. 24, 2014.

(51) Int. Cl. A61F 5/01

(52) U.S. Cl. CPC A61F 5/012; A61F 5/0127; A61F 2250/0003

(57) ABSTRACT

A device and a method to prevent ankle sprain injuries, which are able to detect hazardous situations during malpractice of physical or regular activity and to avoid incidents by deploying an inflatable airbag to block and stabilize the user's ankle joint. A series of sensors monitor the user's ankle motion pattern and provide data to an analyzing part, which indicates whether the movement is hazardous and, upon danger recognition, triggers the inflation of an airbag device to block the user's ankle.

### FIELD OF THE INVENTION

The present invention relates to an ankle airbag device and a method that prevent accidents commonly leading to ankle joint injuries. The device comprises as functional parts at least one inflatable body in proximity of the ankle, an inflating system of release of gas, a series of sensors that monitor ankle motion and electric activity of muscles and an analyzing part configured to evaluate whether the data collected correspond to a sprain motion or another hazardous situation.

### DISCUSSION OF PRIOR ART

Injuries in ligaments and muscles of the ankle are very common amongst athletes of multiple and diverse sports. Injured athletes often need to suspend their activity because of pain and damages, which is certainly saddening for them but can also represent loss of profits for them and their team in competitive disciplines. Additionally, this kind of injuries can lead to irreversible conditions such as ankle instability, which considerably increases risks of recidivism.

Various biomechanical mechanisms can lead to injuries of the ankle joint. But every ankle sprain injury occurs when the foot exceeds certain biomechanical indicators, such as angles and velocities, which can be measured and analyzed by various sensors. Recognizing these excess in time and correcting the foot position through an external stimulus can therefore prevent said injuries.

Different devices have been developed to prevent ankle sprain. Various shoe models covering the ankle joint and hindering ankle inversion movement are available, for example hiking shoes often help preventing perilous movements. Nevertheless this type of shoes cannot offer a high degree of freedom for the user's foot. Remembering that the majority of ankle sprain-related injuries are encountered in sporting activities, this low degree of freedom is very limiting for athlete of said sporting activities using shoes similar to hiking shoes.

Currently, two other devices listed as US20130041427 A1 and CN103182146 respectively have been described. The function of said devices is to prevent sprain ankle injuries. The stimulating part, which is meant to protect the user's ankle and consists of two electrodes that need to be directly applied to the skin, has several disadvantages. First of all its utilization is not practical for the user: the application of the electrodes requires time and, sometimes, a depilated skin surface of the muscles. The use and efficiency of electrodes can be hindered by in-sport conditions such as sudation. Electrical muscular stimulation can also cause skin irritations and burns if not correctly used. Moreover, the response of the muscle might not be strong enough to straighten the ankle, especially if this latter is loaded with the whole body mass of the user. Finally, an incorrect movement of a loaded ankle can result in tendomuscular damages (e.g. lacerations, degree II-III lesions). The method described can be dangerous for the user if the timing is not perfectly respected, in other words the electrical stimulation should occur before the sprain movement even starts, which cannot be done with the sensing part described by the authors.

### SUMMARY OF THE INVENTION

Accordingly, the present invention seeks to reduce the above-identified disadvantages of the prior art by providing an alternative method and device with a higher probability of preventing ankle sprain injuries without causing further damages and with a longer device's autonomy.

In an aspect of the invention a system is provided to prevent ankle injury. It comprises a series of sensors to monitor the ankle motion, an analyzing part configured to evaluate whether the data collected by the sensors correspond to a sprain motion, a trigger device, in communication with the analyzing part, to control the inflation of the airbag and an airbag surrounding the user's ankle that inflates in case of sprain movement to prevent injuries straightening the joint.

In another aspect of the invention a method is provided to prevent ankle injury. It comprises a series of sensors to monitor the ankle motion, an analyzing part configured to evaluate whether the data collected by the sensors correspond to a sprain motion, a trigger device, connected to the analyzing part, to control the inflation of the airbag and an airbag surrounding the user's ankle that inflates in case of sprain movement to prevent injuries straightening the joint.

In another aspect of the invention a shoe containing the above-mentioned device is presented.

It can take the form of a single device, i.e. shoe, or a combination of two or more devices, i.e. shoe, sock, shin guards or other protective elements.

### BRIEF DESCRIPTION OF DRAWINGS

FIG. 1 shows a schematic drawing of the device for ankle injuries prevention according to an embodiment.

FIG. 2 shows a sectional view of the device only composed by a shoe.

FIG. 3 shows a sectional view of the device composed by a shoe and a sock.

### DETAILED DESCRIPTION OF THE INVENTION

The present invention allows the user to diminish the risk of ankle joint injuries caused by ankle sprains by avoiding such movement.

The device detects potentially hazardous situations through the analysis of the data provided by the different sensors and releases the gas into the inflatable bag in order to straighten the user's ankle, blocking any kind of further damaging movement.

Here follows a detailed description of embodiments of the present invention.

A device can include a sensing part, an analyzing part, a trigger part and an inflatable part or airbag.

The sensing part is configured to track and measure the movement of the foot or of the shoe during activity. It comprises one or more sensors that collect information in real time and transmit the data to the analyzing part. The sensors can be accelerometers, tri-axial accelerometers, gyroscopes, tri-axial gyroscopes, electromyography sensors, pressure sensors, inclinometers or tilt sensors or more of the above combined. They can measure various data amongst which angles of plantar flexion, inversion and internal rotation, angular velocities of plantar flexion, inversion and internal rotation, pressure on the foot in motion and muscular activity of the neighboring muscles such as tibialis anterior, peroneus brevis, peroneus longus, soleus, etc.

The sensing part can also include sensors located on the rest of the user's body to monitor the body position and better detect dangerous situations. For example, said sensors can be located on the shoulders, on the hips and on the knees of the user.

Any combination of the above-mentioned sensors can be used in an implementation.

Variations can be introduced to better match a particular sport or discipline by monitoring more hazardous parameter in said sport or discipline. Different mechanisms of ankle sprain have been described and each has its own critical parameters; covering a various set of these parameters is therefore fundamental for a functioning device in multiple disciplines.

In an implementation the device includes a motion sensor 110, composed of a tri-axial accelerometer and a gyroscope, located on the shoe of the user. The position of said sensor can be modified to increase the efficiency of the data collection and the precision of the information collected.

In another implementation the device includes a motion sensor as described above and various pressure sensors 111 located on the sole of the shoe.

In another implementation the sensing part, which can be composed of motion sensors or pressure sensors or both of the above in a shoe, can include electromyography sensors 112 located in the internal part of the shoe or in a sock or both. In this case the device can also include a sock as support.

The data for each of the sensors can be transmitted to the analyzing part 120 through a wire or by wireless connection, e.g. Bluetooth.

The analyzing part can be located anywhere in the shoe, as long as it does not hinder the user's movements, or in an external device (e.g. smartphone app). The position of said part thus depends on the utilization of the device and can be adapted according to, for example, the sporting discipline of the user.

When a situation of danger is detected, the analyzing part sends a trigger signal to the inflator. The analyzing part can transmit said trigger signal once a pre-determined threshold is reached for one or more parameters, when a specific combination of parameters is observed or when a certain trend in one or more parameter is detected. Said triggering situations can be adjusted according to the user's weight, sex, age, sporting discipline or ankle injuries recidivism risk. The analyzing part can also be dynamically adaptive to the user's motion patterns, i.e. the implemented algorithm adapts to the motion of the user through machine learning techniques, and updates of said analyzing part can be provided.

In an embodiment of the present invention the analyzing part 120 is positioned with the sensing part. In another embodiment the said analyzing part is located behind the user's heel. In another embodiment the said analyzing part is not located on the device but is comprised of a software component (e.g. smartphone app, computer program) located on an external device to which the real-time data is transmitted via wireless connection.

The trigger part 130 includes a receiver 131 and an inflator 132. The trigger signal can be transmitted from the analyzing part to the receiver of the trigger part through a wire or by wireless connection. The inflator can have different shapes, for example cylindrical, and it shall be adapted to fit the user's needs in term of size, e.g. small size, and positioning. It can be a pyrotechnic inflator, a cold gas inflator, in which case the device contains a capsule to store the gas, or a hybrid inflator. The inflator is attached to the bag and has a valve to avoid early deflation; it can be stitched, screwed, glued or directly integrated into the bag. Since the quantity of gas needed to inflate the bag is small, approximately 3 liters, the inflator is smaller than most of currently available inflators.

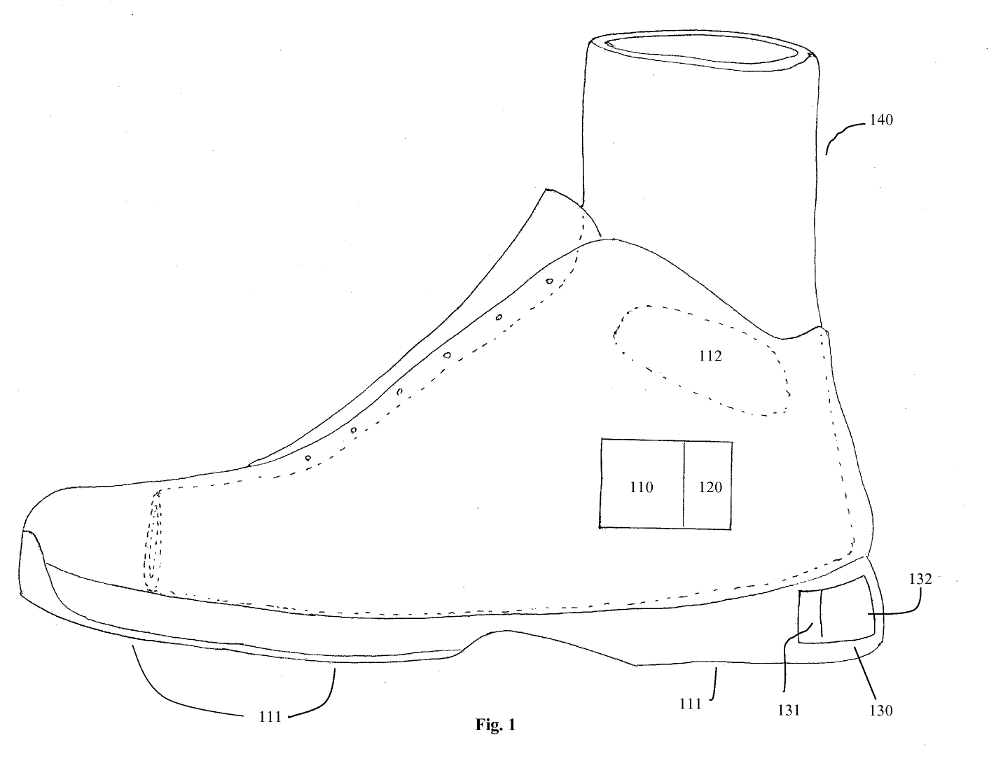

The bag 140 can be made of various materials. Nylon or similar materials are preferred in order to provide the necessary stiffness; alternatively the bag can be made of slightly more permeable materials in order to allow sudation. It should be shaped such as to provide the best stability to the foot once inflated, therefore it should cover more than just the ankle and go high enough on the user's calf and cover most of the user's foot. It can be closed over the toes like a sock or exclude them, as shown in FIG. 1.

The bag can be integrated in the shoe or in a connected sock.

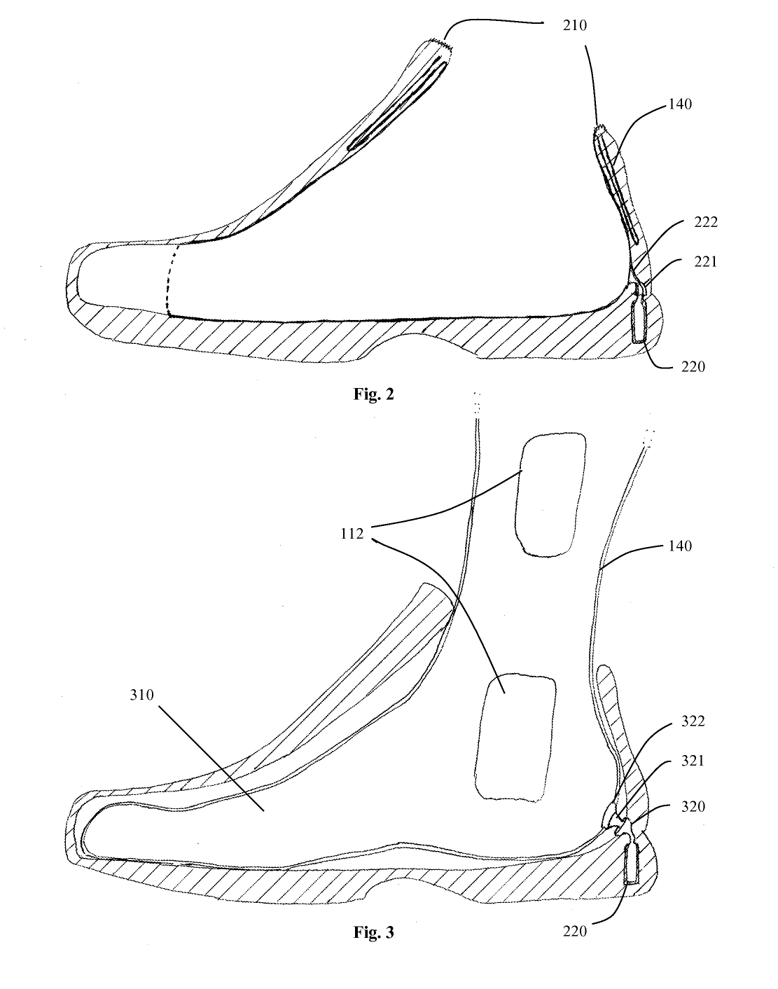

In an embodiment shown in FIG. 2 in which the device is only composed by a shoe, the bag 140 does not cover the user's toes, is folded in order to give the fastest inflation possible and, being thin enough when deflated, is positioned inside the shoe's fabric. For said embodiment to be effective the bag must easily burst some of the seams of the shoe during the inflation process. Said seams 210 are typically located on the upper part of the shoe, near the user's ankle, so that the airbag can reach a higher level on the user's calf to better protect the ankle

Said embodiment's inflator is composed of a capsule 220 to store the gas that is connected to the bag through a valve 231 and a connector 232 which is integrated into the bag.

In another embodiment the airbag 140 covers all the foot and is incorporated in a sock 310. It can be a thin layer in between two fabric layers of said sock. A plugging mechanism 320 is present to ease the use of the device; the sock fits in and gets plugged to the inflator's capsule 220 through a small outlet 321 in order to allow the gas to flow from said inflator into the bag. The part 322 of said plugging mechanism attached to the sock is glued to it. Said embodiment would be more practical for eventual reuses of the sensing shoe.

Said sock can also include the electromyography sensors 112 previously described. Said sensors monitor the muscular activity of various muscles near the ankle joint, depending on the height the sock reaches on the user's calf.

The airbag must be thick enough to provide strong mechanical support to the failing ankle A series of internal seams can be used to give the most suitable shape and structure to the airbag in order to increase its mechanical strength and lower the pressure it exerts on the foot of the user.

The inflation of the bag must happen quickly enough to prevent any major injury. Since the critical moments for ankle injuries happen in less than one second the airbag can also include a deflating mechanism to release the pressure on the user's foot.

The present invention has been described herein above in various embodiments, however it is not limited to them exclusively. The invention extends to any novel one or novel combination of one or more of the above-described features and to any novel or novel combination of the processes for the described method. Variations and modifications can be made and the said invention's utilization is not solely related to sport practice.

The invention claimed is:

1. An ankle airbag device for protecting the user from sprain injuries, said device comprising:
a sensing part to monitor the user's ankle motion;
an analyzing system configured to evaluate whether the data collected by the sensors correspond to a sprain motion;
a trigger device to activate the inflating system upon signal of the analyzing system;
at least one inflatable body in proximity of the ankle;
an inflating system for said inflatable body; and
a trigger device in communication with the analyzing system to control the inflation of said inflatable body.

2. The ankle airbag device of claim 1, wherein the said device is integrated in a shoe so not as to hinder the user's movements.

3. The ankle airbag device of claim 1, wherein the said device is a combination of two or more devices, integrated in a shoe, a sock, another sporting gear located near the ankle, or any combination thereof and containing the different parts.

4. The ankle airbag device of claim 1, wherein the sensing part is composed of one or more of the following sensors in any combination: accelerometers, tri-axial accelerometers, gyroscopes, tri-axial gyroscopes, electromyography sensors, pressure sensors, inclinometers or tilt sensors.

5. The ankle airbag device of claim 4, wherein the sensors are located on the device as well as on the rest of the user's body.

6. The ankle airbag device of claim 1, wherein the sensing part is composed of a tri-axial accelerometer and a gyroscope located on the shoe.

7. The ankle airbag device of claim 6, wherein the sensing part is completed by various pressure sensors located in the sole of the shoe and by electromyography sensors integrated in the device to sense the activity of the neighboring muscles.

8. The ankle airbag device of claim 1, wherein the analyzing part is located on the device and receives the data from the sensing parts through wires or by wireless connection

9. The ankle airbag device of claim 1, wherein the analyzing part is partially located on the device under the form of a transmitter and partially located on an external device for the computational tasks, and receives the data from the sensing parts through wires or by wireless connection.

10. The ankle airbag device of claim 1, wherein the analyzing part sends a signal to the trigger device upon reaching a pre-determined threshold, characterized by the value of a parameter or the combination of several parameters.

11. The ankle airbag device of claim 10, wherein the threshold is adapted according to the user's characteristics such as weight, sex, age, sporting disciplines, ankle injury recidivism risk or a combination thereof and is adaptive to the user's motion patterns via machine learning mechanisms.

12. The ankle airbag device of claim 1, wherein the trigger device is composed of a receiver and an inflator and induces the inflation of one or more inflating bodies upon reception of the signal from the analyzing part.

13. The ankle airbag device of claim 12, wherein the inflator is directly connected to the inflatable body through a plugging mechanism and belongs to one of the following types: pyrotechnic inflators, cold gas inflators or hybrid inflators.

14. The ankle airbag device of claim 1, wherein the inflatable body is an airbag integrated in the fabric of the shoe, is folded in order to give the fastest inflation possible, and is positioned such as it can easily burst seams on the upper side of the shoe so as to reach a higher level on the user's calf upon inflation.

15. The ankle airbag device of claim 1, wherein the inflatable body is an airbag integrated in a disposable sock, which can be connected to the inflator through a plugging mechanism.

16. The ankle airbag device of claim 1, wherein the inflatable body is an airbag reinforced with internal seams so as to have the optimal shape and rigidity for ankle stabilization.

17. A method for protecting the user from sprain injuries, said method including:
monitoring user's ankle motion;
analyzing real-time data and establishing whether it corresponds to a sprain motion;
triggering a device to inflate an airbag to stabilize the user's ankle

18. The ankle sprain prevention method of claim 17, wherein the user's motion is monitored by sensing one or more of the following data: angles of plantar flexion, inversion and internal rotation, angular velocities of plantar flexion, inversion and internal rotation, pressure on the foot in motion, and muscular activity of the neighboring muscles such as tibialis anterior, peroneus brevis, peroneus longus, and soleus.

19. The ankle sprain prevention method of claim 17, wherein the triggering occurs upon reaching a pre-determined threshold during the analysis, said threshold being characterized by the value of a parameter or the combination of several parameters and being adaptive to the user's motion patterns.

20. The ankle sprain prevention method of claim 19, wherein the threshold is adapted according to the user's characteristics such as weight, sex, age, sporting disciplines, ankle injury recidivism risk or a combination thereof.
